(19) United States
(12) Patent Application Publication
(10) Pub. No.: US 2010/0166739 A1
(43) Pub. Date: Jul. 1, 2010

(54) Methods and Compositions for Diagnosing Urological Disorders

(75) Inventors: Michael B. Chancellor, Pittsburgh, PA (US); Hann-Chorng Kuo, Hualien (TW)

Correspondence Address: Pabst Patent Group LLP, 1545 PEACHTREE STREET NE, SUITE 320, ATLANTA, GA, 30309, US

(73) Assignee: Lipella Paharmaceuticals Inc.

(21) Appl. No.: 12/649,686

(22) Filed: Dec. 30, 2009

Related U.S. Application Data
(60) Provisional application No. 61/141,514, filed on Dec. 30, 2008.

(51) Int. Cl. A61K 39/395; A61K 39/00; G01N 33/53; G01N 33/00; A61K 31/7088; A61P 13/00

(52) U.S. Cl. 424/130.1; 424/184.1; 435/7.1; 436/86; 514/44 A

(57) ABSTRACT

It has been discovered that cytokines, chemokines, and growth factors in urine are biomarkers indicative of urological disorders including interstitial cystitis/painful bladder syndrome and overactive bladder syndrome. Preferred chemokine biomarkers are CCL2, CCL4 (MIP-1β), CCL11, CXCL1 (GRO-α), sCD40L, IL-12p70/p40, IL-5, sIL-2Rα, IL-6, IL-10, IL-8, and EGF. The concentration of one or more of these chemokines in an urine sample can be used to assist in the diagnosis of urological disorders. Methods for evaluating the effectiveness of treatments for urological disorders and for assessing the severity of urological disorders are also provided.

### CROSS-REFERENCE TO RELATED APPLICATIONS

This application claims priority to and benefit of U.S. Patent Application No. 61/141,514 filed on Dec. 30, 2008, and where permissible is incorporated by reference in its entirety.

### FIELD OF THE INVENTION

The invention is generally related to the field of urology and the detection, diagnosis, or treatment of urological disorders.

### BACKGROUND OF THE INVENTION

Urological disorders such as interstitial cystitis/painful bladder syndrome (IC/PBS) and overactive bladder (OAB) syndrome can be difficult to diagnose and treat. In many cases, objective information about the disease is only available through expensive and invasive procedures such as cystoscopy and tissue biopsy (Erickson, D. R., et al., J Urol, 173:93 (2005)).

Interstitial Cystitis/Painful Bladder Syndrome (IC/PBS)

Symptoms of interstitial cystitis vary and some people may experience mild discomfort, pressure, tenderness, or intense pain in the bladder and pelvic area. Symptoms may or may not include an urgent need to urinate, frequent need to urinate, or a combination of these symptoms. Pain may change in intensity as the bladder fills with urine or as it empties. Women's symptoms often get worse during menstruation.

The bladder wall of the patient suffering from interstitial cystitis may be irritated and become scarred or stiff. Glomerulations (pinpoint bleeding caused by recurrent irritation) may appear on the bladder wall. The bladders of some interstitial cystitis sufferers have decreased urine capacity, which increases the frequency of the need to urinate. Frequency, however, is not always specifically related to bladder size. Many patients with severe frequency have normal bladder capacity. Patients with severe cases of interstitial cystitis may urinate as much as 60 times a day.

Interstitial cystitis is a poorly understood disease with unknown causes. Although no bacteria, viruses or other pathogens have been found in the urine of interstitial cystitis sufferers, an unidentified infectious agent may be the cause. There is no definitive test to identify interstitial cystitis. Because symptoms are similar to those of other disorders of the urinary system such as urinary tract or vaginal infections, bladder cancer, bladder inflammation or infection caused by radiation to the pelvic area, eosinophilic and tuberculous cystitis, kidney stones, endometriosis, neurological disorders, sexually transmitted diseases, low-count bacteria in the urine, and, in men, chronic bacterial and nonbacterial prostatitis, diagnosis is sometimes difficult.

The diagnosis of interstitial cystitis in the general population is based on presence of urgency, frequency, or pelvic/bladder pain cystoscopic evidence (under anesthesia) of bladder wall inflammation, including Hunner's ulcers or glomerulations (present in 90 percent of patients with interstitial cystitis) and the absence of other diseases that could cause the symptoms.

Diagnostic tests that help identify other conditions include urinalysis, urine culture, cystoscopy, biopsy of the bladder wall, distention of the bladder under anesthesia, urine cytology, and, in men, laboratory examination of prostate secretions.

Overactive Bladder Syndrome (OAB)

Overactive bladder (OAB) syndrome affects more than 17 million people in the United States. OAB is characterized by lower urinary tract symptoms of urgency, frequency, and/or urgency incontinence (Tyagi, et al., Urol. Clin. North Am., 33:433-438 (2006)). OAB often associated with detrusor overactivity, a pattern of bladder muscle contraction observed during urodynamics, and with OAB are often treated for detrusor overactivity.

There is a dearth of information available in art on the etiological basis of OAB, but various other conditions affecting the urinary tract have been linked to biomarkers of inflammation (Steers, et al., BJU Int., 85(Suppl. 3):69: 70-71 (2000), Segerer, et al., Scientif: World J., 5:835-844 (2005), Tesch, et al., Am. J Physiol. Renal Physiol., 294:F687-F701 (2008), Ferri, et al., Eur. Rev. Med. Pharma. Sci., 11:171-178 (2007), Bagshaw, et al., Intensive Care Med., 33:1285-96 (2007), Li, et al., Autoimmun. Rev., 5:383-388 (2006)). Biopsy studies have demonstrated physical signs of inflammation in OAB in the absence of UTI (Loran, et al., Urologiia, 2:37-41 (2007)). Inflammation in the bladder typically involves lymphocytic and monocytic infiltrates restricted to the upper layers of the bladder wall, especially, the sub-urothelium (Comperat, et al., Eur. Urol., 50:1058-64 (2006)). Studies suggest that bladder inflammation induced by infiltrating immune cells can be further amplified by the resident cells present in both the urothelium and detrusor through the release of chemokines such as CCL2 and IL-8 (Bouchelouche, et al., J. Urol., 171:462-66 (2004), Bouchelouche, et al., Urology, 67:214-219 (2006)).

Urgency is the core symptom indicating the presence of OAB, and the use of an urgency perceptive scale or urgency severity score has been suggested (Abrams, et al., BJU Int. 96(Suppl. 1):1-3 (2005)). Symptom score instruments are based on subjective reporting by the patients who must grade their own degree of urgency. The subjective nature of this scoring scale may account for the wide variation among reported grades of urgency severity (Nixon, et al., J. Urol., 174:604-7 (2005)).

There are inherent disadvantages in biopsy-based diagnoses, for example, the biopsy procedure is invasive, expensive, associated with morbidity, and highly variable due to the small sample size relative to the entire bladder. In the case of interstitial cystitis, a biopsy may only be diagnostic in patients with end-organ disease. Therefore, a need exists to develop to objective, quantitative, and non-invasive methods for diagnosing urological disorders. Development of biomarkers for diagnosis and prediction of urological disorder progression is therefore a high priority.

Thus, it is an object of the present invention to provide biomarkers for the diagnosis of urological disorders.

It is another object of the present invention to provide methods for diagnosing or assisting in the diagnosis of an urological disorder.

It is a further object of the present invention to provide methods for diagnosing or assisting in the diagnosis of interstitial cystitis/painful bladder syndrome.

It is another object of the present invention to provide methods for diagnosing or assisting in the diagnosis of overactive bladder (OAB) syndrome.

It is a further object of the present invention to provide methods for monitoring progression of urological disorders.

It is another object of the present invention to provide methods for distinguishing between two urological disorders.

It is still another object of the present invention to provide methods and compositions for treating one or more symptoms of an urological disorder.

### SUMMARY OF THE INVENTION

It has been discovered that chemokines relevant to chemotaxis of eosinophils, chemotaxis of monocytes, and activation of mast cells in urine are biomarkers indicative of urological disorders including interstitial cystitis/painful bladder syndrome. Preferred inflammatory biomarkers are chemokines, such as CCL2, CCL4 (MIP-1β), CCL11 (eotaxin), and CXCL1 (GRO-α); cytokines such as sCD40L, IL-12p70/p40, IL-5, sIL-2Rα, IL-6, IL-10, and IL-8; and growth factors such as epidermal growth factor (EGF), and combinations thereof. The concentration of one or more of these biomarkers in an urine sample can be used to assist in the diagnosis of urological disorders. A preferred urological disorder to diagnose is interstitial cystitis/painful bladder syndrome (IC/PBS). Another preferred urological disorder is overactive bladder syndrome (OAB).

A method for diagnosing or assisting in the diagnosis of an urological disorder includes assaying an urine sample for concentrations of biomarkers including chemokines such as CCL2, CCL4 (MIP-1β), CCL11, and CXCL1 (GRO-α); cytokines such as sCD40L, IL-12p70/p40, IL-5, sIL-2Rα, IL-6, IL-10, and IL-8; and growth factors such as epidermal growth factor (EGF), and combinations thereof, wherein elevated concentrations of one or more of the biomarkers relative to a normal control is indicative of an urological disorder. In a preferred embodiment, biomarkers are chemokines relevant to chemotaxis of eosinophils, chemotaxis of monocytes, and activation of mast cells. The method optionally includes the step of recording or reporting a diagnosis of the urological disorder or generating a diagnosis report. The report typically includes the concentrations of one or more of CCL2, CCL4 (MIP-1β), CCL11, CXCL1 (GRO-α), sCD40L, IL-12p70/p40, IL-5, sIL-2Rα, IL-6, IL-10, IL-8, EGF and/or combinations thereof compared to control or reference concentrations. In one embodiment, only CCL2 is assayed.

Another method for assessing the severity of an urological disorder is by assaying an urine sample for concentrations of biomarkers wherein the concentrations of the biomarkers are correlated to the severity of an urological disorder. The method optionally includes the step of reporting or recording the severity of the urological disorder based on the concentrations of the biomarkers. The degree of elevation of the concentrations of chemokines in the urine sample relative to a control are indicative of increased severity of the urological disorder, for example interstitial cystitis/painful bladder syndrome or overactive bladder syndrome. In one preferred embodiment, IL-8 levels are measured.

Another method assesses the effectiveness of a treatment for an urological disorder by assaying an urine sample for concentrations of biomarkers from a subject undergoing treatment for the urological disorder, wherein elevated concentrations of the chemokines in the urine sample from the subject relative to a control or to a pre-treatment baseline indicates the treatment for the urological disorder is ineffective or sub-optimal. The method optionally includes the step of recording or reporting the effectiveness of the treatment based on the concentrations of the biomarkers in the urine sample.

Still another embodiment provides a kit designed for facilitating the diagnosis of an urological disorder, such as IC/PBS or OAB. The kit includes reagents for detecting, in urine, one or more biomarkers such as CCL2, CCL4 (MIP-1β), CCL11, CXCL1 (GRO-α), sCD40L, IL-12p70/p40, IL-5, sIL-2Rα, IL-6, IL-10, IL-8, and EGF and a sample container for holding an urine sample. In a preferred embodiment, the kit includes a means for detecting, in urine, one or more of CCL2, CCL4, and CCL11. The kit also includes instructions for use. In one embodiment the means for detecting one or more of the protein biomarkers comprises an antibody-based binding moiety that specifically binds to the biomarkers to be detected. The kit components are packaged in a container.

Another embodiment provides a method for treating an urological disorder by administering an effective amount of an antagonist of a chemokine or cytokine. Preferred antagonists are antagonists to chemokines relevant to chemotaxis of eosinophils, chemotaxis of monocytes, and activation of mast cells to reduce inflammation in bladder relative to a control. Representative chemokine antagonists include, but are not limited to, anti-chemokine antibodies or antigen-binding fragments thereof, small molecule antagonists, inhibitory nucleic acids and combinations thereof.

### BRIEF DESCRIPTION OF THE DRAWINGS

FIG. 1 is a graph comparing urine levels for the chemokine CCL2 in patients having severe interstitial cystitis/painful bladder syndrome (IC/PBS) (▾), moderate IC/PBS (▴), and a control group (▪).

FIG. 2 is a graph comparing urine levels for the chemokine MIP-1β (CCL4) in patients having severe IC/PBS (▾), moderate IC/PBS (▴), and a control group (▪).

FIG. 3 is a graph comparing urine levels for the chemokine eotaxin (CCL11) in patients having severe IC/PBS (▾), moderate IC/PBS (▴), and a control group (▪).

FIG. 4 is a graph comparing urine levels [pg/mg creatinine] for the cytokine IL-8 in patients having severe interstitial cystitis/painful bladder syndrome (IC/PBS) (), mild IC/PBS (▪), and a control group (▴).

FIG. 5 is a bar graph comparing urine levels [pg/mg creatinine, log scale] for biomarkers [left to right: IL-12p70/p40s, MIP-1β (CCL4), GRO-α (CXCL1), CCL2, IL-5, sCD40L, IL-10, sIL-2Rα, and epidermal growth factor (EGF)] in control individuals (black bars) and patients with overactive bladder syndrome (white bars).

FIG. 6 A is a graph comparing urine levels [pg/mg creatinine] of CCL2 in asymptomatic, control individuals (▪) and in patients having overactive bladder syndrome (OAB) (▴). FIG. 6 B is a graph comparing urine levels [pg/mg creatinine] for MIP-1β in asymptomatic, control individuals (▪) and in patients having overactive bladder syndrome (OAB) (▴). FIG. 6 C is a graph comparing urine levels [pg/mg creatinine] for IL-5 in asymptomatic, control individuals (▪) and in patients having overactive bladder syndrome (OAB) (▴). FIG. 6 D is a graph comparing urine levels [pg/mg creatinine] for sIL-2Ra in asymptomatic, control individuals (▪) and in patients having overactive bladder syndrome (OAB) (▴).

### DETAILED DESCRIPTION OF THE INVENTION

### I. Definitions

The term “test sample” refers to an urine sample obtained from a subject being tested for an urological disorder. Representative urological disorders include, but are not limited to, interstitial cystitis/painful bladder syndrome, overactive bladder syndrome, urinary tract or vaginal infections, bladder cancer, bladder inflammation or infection, eosinophilic and tuberculous cystitis, kidney stones, endometriosis, low-count bacteria in the urine, and, in men, chronic bacterial and nonbacterial prostatitis.

The term “control sample” refers to an urine sample obtained from a different subject who is asymptomatic or does not have an urological disorder such as interstitial cystitis/painful bladder syndrome (also known as IC/PBS) or overactive bladder syndrome (also known as OAB).

The term “a reference level” refers to a level of biomarker protein that is present in a subject who is asymptomatic for an urological disorder such as interstitial cystitis/painful bladder syndrome or overactive bladder syndrome.

The “reference level” can be an average level of a biomarker protein, e.g. obtained from data from multiple subjects. Alternatively, a test sample can be compared directly to the level present in a control sample. For purposes of comparison, the biomarker of the test sample is compared to a reference level for the same biomarker protein. A reference level can be higher or lower than the sample being tested. A “positive” control is a value for individuals diagnosed with the disease or disorder to be tested for; a “normal” control is a value for individuals who do not have the disease or disorder to be tested for.

When discriminating between two disorders, test samples may be compared to reference levels of patients previously diagnosed with an urological disorder such as IC/PBS or OAB.

The term “a higher level of biomarker protein in the test sample as compared to the level in the control sample” refers to an amount of biomarker protein that is greater than an amount of biomarker protein present in a control sample. The term “higher level” refers to a level that is statistically significant or significantly above levels found in the control sample. Preferably, the “higher level” is at least 2 fold greater.

The term “a lower level of biomarker protein in the test sample as compared to the level in the control sample” refers to an amount of biomarker protein that is lower than an amount of biomarker protein present in a control sample. The term “lower level” refers to a level that is statistically significant or significantly below levels found in the control sample. Preferably, the “lower level” is at least 2 fold less.

The term “biomarker protein” or “biomarker” refers to inflammatory chemokines, cytokines, and growth factors including, but not limited to, CCL2, CCL4 (MIP-1β), CCL11, CXCL1 (GRO-α), sCD40L, IL-12p70/p40, IL-5, sIL-2Ra, IL-6, IL-10, IL-8, and EGF.

The term “statistically significant” or “significantly” refers to statistical significance and generally means a two standard deviation (2SD) above or below the concentration of the reference level.

“Specific binding” between a binding agent, e.g., an antibody and a protein, for instance, a biomarker, refers to the ability of a capture- or detection-agent to preferentially bind to a particular molecule that is present in a mixture; e.g., a biological sample. Specific binding refers to a dissociation constant (K_{D}) that is less than about 10⁻⁶ M; preferably, less than about 10⁻⁸ M; and, most preferably, less than about 10⁻⁹ M.

The phrase “specifically (or selectively) binds” to an antibody or “specifically (or selectively) immunoreactive with” when referring to a protein or peptide, refers to a binding reaction that is determinative of the presence of the protein in a heterogeneous population of proteins and other biologics. For example, under designated immunoassay conditions, the specified antibodies bind to a particular protein or protein complex at least two times the background and do not substantially bind in a significant amount to other proteins present in the sample.

### II. Biomarkers for Identifying Urological Disorders

It has been discovered that elevated concentrations of biomarkers involved in inflammation and tissue repair are indicative of urological disorders. Examples of inflammatory biomarkers that serve as urinary biomarkers for urological disorders and disease progression include, but are not limited to, chemokines such as CCL2 (MCP-1), CCL4 (MIP-1β), CCL11, and CXCL1 (GRO-α); cytokines such as sCD40L, IL-12p70/p40, IL-5, sIL-2Rα, IL-6, IL-10, and IL-8; growth factors such as epidermal growth factor (EGF), and combinations thereof. Levels of inflammatory biomarkers in the urine are in comparison to levels of the same inflammatory biomarkers in urine samples from one or more subjects that do not have an urological disorder. Levels of inflammatory biomarkers in the urine are also an indicator of the severity of the urological disorder. Levels of inflammatory biomarkers can also be used to distinguish between different urological diseases.

The disclosed methods may rely on one biomarker or combinations of biomarkers. For example, in one embodiment, the method employs more than one chemokine. In another embodiment, the method employs one or more chemokines, cytokines, and growth factors in combination. Preferred biomarkers for diagnosing or assisting in the diagnosis of interstitial cystitis/painful bladder syndrome are chemokines relevant to chemotaxis of eosinophils, chemotaxis of monocytes, and activation of mast cells such as CCL2, CCL4, and CCL11. In another embodiment the biomarker is IL-8. Preferred biomarkers for diagnosing or assisting in the diagnosis of overactive bladder syndrome are CCL2, CCL4 (MIP-1β), CXCL1 (GRO-α) sCD40L, IL-12p70/p40, IL-5, sIL-2Rα, IL-6, and IL-10, and EGF.

A. Chemokine Biomarkers

Chemokines constitute a large family of secretory proteins that are expressed by leukocytes as well as non-inflammatory cells in the bladder (Bouchelouche, et al., J. Urol., 171:462-66 (2004), Bouchelouche, et al., Urology, 67:214-219 (2006)). Chemokines are subdivided into four families (CXC, CC, C, and CX3C) based on the relative position and the number of conserved N-terminal cysteine residues as well as the absence (CC) or presence of intervening amino acid(s) between the cysteine residues (CXC). The CC class of chemokines, such as CCL2, provokes mast cell activation and has chemotactic activity for monocytes, but not for neutrophils. The CXC chemokines, with the glutamic acid-leucine-arginine motif preceding the first cysteine, are known to be angiogenic and promote neutrophil chemotaxis (e.g., GRO-α/CXCL1) (Moser, et al., J. Biol. Chem., 266:10666-71 (1991)). Preclinical studies suggest that increased urine levels of T_{H}2 polarizing factor CCL2 and CXCL1 accompany bladder inflammation, and elevated seminal plasma levels of IL-8 have been linked to the symptoms of chronic prostatitis/chronic pelvic pain syndrome and nonspecific urethritis (Smaldone, et al., Urology, 73(2):421-26 (2009), Khadra, et al., BJU Int., 97:1043-46 (2006)).

Chemokines not only induce chemotaxis but also activation of these target cells in the bladder and contribute to inflammatory-induced changes of rheumatoid arthritis, Crohn's disease and IC/PBS (Segerer, S., Nelson, P. J., Scientific World Journal, 5: 835 (2005)). Preferred chemokines that serve as urinary biomarkers for urological disorders include, but are not limited to, chemokines relevant to chemotaxis of esosinophils, chemotaxis of monocytes, and activation of mast cells. Representative chemokines that can be used as biomarkers for diagnosing or assisting in the diagnosis of urological disorders include, but are not limited to, monocyte chemotactic proteins, macrophage inflammatory proteins, and eotaxins. Most preferred chemokines are CCL2, CCL4 (MIP-1β), CCL11, and CXCL1 (GRO-α), and combinations thereof. These chemokines can be used individually or in combination as biomarkers for urological disorders, in particular inflammatory disorders of the bladder such as interstitial cystitis/painful bladder syndrome and overactive bladder syndrome. Preferred chemokine biomarkers of interstitial cystitis/painful bladder syndrome are CCL2, CCL4 (MIP-1β), and CCL11. Preferred chemokine biomarkers for overactive bladder syndrome are CCL2, CCL4 (MIP-1β), and CXCL1 (GRO-α). These biomarkers can also be used to monitor the progression of urological disorders or determine the effectiveness of a treatment for urological disorders by monitoring the concentrations of these biomarkers in urine samples of patients undergoing treatment for an urological disorder.

1. Macrophage Inflammatory Proteins

One embodiment provides a method for diagnosing or assisting in the diagnosis of an urological disorder by determining the concentration of macrophage inflammatory proteins (MIP) in an urine sample from a subject and comparing the concentration of MIP in the urine sample to the concentration of MIP in the urine sample of a control, for example a subject that does not have an urological disorder. Concentrations of MIP in urine that are higher than the control or reference level of MIP are indicative of an urological disorder, for example, interstitial cystitis/painful bladder syndrome or overactive bladder syndrome.

A preferred MIP is MIP-1β. In humans, there are two major fowls of MIP, MIP-1a and MIP-1β, now officially named CCL3 and CCL4, respectively. Both are major factors produced by macrophages after they are stimulated with bacterial endotoxins (Sherry, et al., J. Exp. Med., 168: 2251-2259 (1988)). They activate human granulocytes (neutrophils, eosinophils and basophils) which can lead to acute neutrophilic inflammation. They also induce the synthesis and release of other pro-inflammatory cytokines such as interleukin 1 (IL-1), IL-6 and TNF-α from fibroblasts and macrophages. MIP-1β is released from mast cells and macrophages can bind heparin and have inflammatory and neutrophil chemokinetic properties (Torrence, A. E., et al., Inflamm Bowel Dis, 14(4):480-90 (2007)).

The polypeptide sequence for CCL3 and CCL4 are known in the art. See Genbank Accession Nos. NP002974 and CAG46916, respectively.

2. Monocyte Chemotactic Protein

One method for diagnosing or assisting in the diagnosis of an urological disorder is by determining the concentration of monocyte chemotactic protein-1 (MCP-1, also known as CCL2) in an urine sample of a subject and comparing the concentration of CCL2 in the urine sample to the concentration of CCL2 in the urine sample of a control, for example a subject that does not have an urological disorder. Concentrations of CCL2 in urine that are higher than the control or reference level of CCL2 are indicative of an urological disorder, for example, interstitial cystitis/painful bladder syndrome or overactive bladder syndrome.

CCL2 is a small cytokine belonging to the CC chemokine family that is also known as monocyte chemotactic protein-1 (CCL2). CCL2 recruits monocytes, memory T cells, and dendritic cells to sites of tissue injury and infection (Carr, M. W., et al., Proc. Natl. Acad. Sci. U.S.A., 91(9):3652-6 (1994); Xu, L. L., et al., J. Leukoc. Biol., 60(3):365-71 (1996)). CCL2 provokes mast cell activation and has chemotactic activity for monocytes that matures into macrophages at site of inflammation (Bouchelouche, K., et al., J Urol, 171: 462 (2004)). This chemokine is produced as a protein precursor containing signal peptide of 23 amino acids and a mature peptide of 76 amino acids (Yoshimura, T., et al., FEBS Lett., 244 (2):487-93 (1989); Furutani, Y., Biochem. Biophys. Res. Commun., 159(1):249-55 (1989)). It is a monomeric polypeptide, with a molecular weight of approximately 13 kDa. The cell surface receptors that bind CCL2 are CCR2 and CCR4 (Craig, M. J., et. al., Cancer Metastasis Rev., 25(4):611-9 (2006)). The polypeptide sequence for CCL2 is known in the art. See Genbank Accession No. AAH09716.

3. Eotaxins

One embodiment provides a method for diagnosing or assisting in the diagnosis of an urological disorder by determining the concentration of CCL11 in an urine sample of a subject and comparing the concentration of CCL11 in the urine sample to the concentration of CCL11 in the urine sample of a control, for example a subject that does not have an urological disorder. Concentrations of CCL11 in urine that are higher than the control or reference level of CCL11 are indicative of an urological disorder, for example interstitial cystitis/painful bladder syndrome.

Chemokine (C-C motif) ligand 11 (CCL11) is a small cytokine belonging to the CC chemokine family that is also known as eotaxin-1. CCL11 selectively recruits eosinophils by inducing their chemotaxis, and therefore, is implicated in allergic responses (Ponath, RD., et al., J. Clin. Invest., 97(3):604-12 (1996); Garcia-Zepeda, E. A., et al., Nat. Med., 2(4):449-56 (1996)). The effects of CCL11 are mediated by its binding to a G-protein-linked receptor known as a chemokine receptor. Chemokine receptors for which CCL11 is a ligand include CCR2, (Ogilvie, P., et al., Blood, 97(7):1920-4 (2001)) CCR3 (Kitaura, M., J. Biol. Chem., 271(13):7725-30 (1996)) and CCR5 (Ogilvie, P., et al., Blood, 97(7):1920-4 (2001)). However, it has been found that eotaxin-1 (CCL11) has high degree selectivity for its receptor, such that they are inactive on neutrophils and monocytes, which do not express CCR3 (Baggiolini, M., et al., Annu. Rev. Immunol., 15:675-705 (1997).

Additional eotaxin chemokines that can be used an urinary biomarkers for urological disorders include, but are not limited to, CCL24, also known as eotaxin-2, and CCL26, also known as eotaxin-3.

The polypeptide sequences for CCL11, CCL24, and CCL26 are known in the art. See Genbank Accession Nos. CAG33702, EAW71772, and EAW71771.

4. Growth Related Oncogene α

One embodiment provides a method for diagnosing or assisting in the diagnosis of an urological disorder by determining the concentration of Growth Related Oncogene a (GRO-α, also known as CXCL1) in an urine sample of a subject and comparing the concentration of CXCL1 in the urine sample to the concentration of CXCL1 in the urine sample of a control, for example a subject that does not have an urological disorder. Concentrations of CXCL1 in urine that are higher than the control or reference level of CXCL1 are indicative of an urological disorder, for example overactive bladder syndrome.

Chemokine (C-X-C motif) ligand 1 (CXCL1) is a small cytokine, chemoattractant with mitogenic properties that prodominental stimulates neutrophils. CXCL1 is secreted by macrophages, neutrophils, epithelial cells, and some cancer cells including human melanoma cells (lida, et al., Mol. Cell. Biol., 10(10): 5596-5599 (1990). It binds with high affinity to IL-8R2, but not IL-8R1 (Moser, et al., Biochem. J., 294, 285-292 (1993). The polypeptide sequence for CXCL1 is known in the art. See Genbank Accession No. NP001502.

B. Cytokines

It has also been discovered that elevated concentrations of cytokines in urine are indicative of urological disorders, in particular inflammatory disorders of the bladder such as interstitial cystitis/painful bladder syndrome and overactive bladder syndrome. Representative cytokines that can be used as biomarkers for diagnosing or assisting in the diagnosis of urological disorders include, but are not limited to, sCD40L, IL-12p70/p40, IL-5, sIL-Ra, IL-6, IL-10, and IL-8. These cytokines can be used individually or in combination as biomarkers for urological disorders, in particular inflammatory disorders of the bladder such as interstitial cystitis/painful bladder syndrome and overactive bladder syndrome. These biomarkers can also be used to monitor the progression of urological disorders or determine the effectiveness of a treatment for urological disorders by monitoring the concentrations of these cytokines in urine samples of patients undergoing treatment for an urological disorder. As demonstrated in Example 2 below, a preferred cytokine for monitoring the progression of an urological disease is IL-8.

1. sCD40L

One embodiment provides a method for diagnosing or assisting in the diagnosis of an urological disorder by determining the concentration of sCD40L in an urine sample of a subject and comparing the concentration of sCD40L in the urine sample to the concentration of sCD40L in the urine sample of a control, for example a subject that does not have an urological disorder. Concentrations of sCD40L in urine that are higher than the control or reference level of sCD40L are indicative of an urological disorder, for example overactive bladder syndrome.

CD40L, also known as CD154, is a trimeric 33-10a type II membrane glycoprotein that is predominantly expressed by activated T and B cells, astrocytes and platelets. CD40L also occurs in a soluble, secreted form (sCD40L) that retains biological activity to bind and activate membrane-bound CD40. Soluble CD40 ligand (sCD40L) is contained in platelet granules (Giunta, et al., Drugs Future, 34(4):333-340 (2009)). When released, sCD40L binds to CD40 receptors, such as those found on endothelial and smooth muscles cell, and initiates release of inflammatory mediators, increases activity of matrix metalloproteinases, and activates the coagulation cascade. The polypeptide sequence for CD40L is known in the art. See Genbank Accession No. NP000065.

2. Interleukins and Soluble Interleukin Receptors

Another method for diagnosing or assisting in the diagnosis of an urological disorder is by determining the concentration of one or more interleukins or soluble interleukin receptors in an urine sample of a subject and comparing the concentration of the one or more interleukins or soluble interleukin receptors in the urine sample to the concentration of the same one or more interleukins or soluble interleukin receptors in the urine sample of a control, for example a subject that does not have an urological disorder. Concentrations of one or more interleukins or soluble interleukin receptors in urine that are higher than the control or reference level (obtained from one or more individuals, typically an average of several individuals) of the same one or more interleukins or soluble interleukin receptors are indicative of an urological disorder, for example overactive bladder syndrome. In preferred embodiments, the interleukin is IL-12, IL-5, IL-6, IL-10, IL-8, sIL-2Ra or variants or combinations thereof. In the most preferred embodiments, the interleukin is IL-12 or IL-5 is used to diagnose or assist in diagnosing overactive bladder syndrome.

Interleukins are a group of proteins belonging to the cytokine family. They have numerous activities which regulate the inflammatory response and the immunological response, however, their major role is the development and differentiation of T and B lymphocytes and hematopoietic cells. The majority of interleukins are synthesized by helper CD4+T lymphocytes, as well as through monocytes, macrophages, and endothelial cells.

Interleukins can consist of one or more polypeptides. For example, IL-12 is comprised of independently-regulated disulfide-linked 40 kDa (p40) and 35 kDa (p35) subunits. The p40 subunit exists extracellularly as a monomer or dimer and can antagonize the action of IL-12p70 (Klinke, Metabolic Engineering and Systems Biology Poster Session (2006)). The polypeptide sequences for interleukins are known in the art. See for example, Genebank Accession Nos. AAD56386 (IL-12, p40), AAD56386 (IL-12, p35), NP000870 (IL-5), AA104253 (IL-10), AAH13615 (IL-8), and NP000591 (IL-6).

Secreted interleukins bind to interleukin receptors, which may be membrane bound or soluble. For example, IL-2 is secreted by CD4+T, which binds to IL-2 receptors and stimulates the growth, differentiation and survival of antigen-selected cytotoxic T cells via the activation of the expression of specific genes. Due to the action of protein lyase at special sites, membrane IL-2R can be partly incised and become free in blood, forming the soluble IL-2R (Shi, et al., World J. Gastroent., 10(24):3674-3676 (2004)). Soluble IL-2R can block IL-2R and thus promote the growth of tumors and parasites. Therefore, in some embodiments, elevated levels of soluble interleukin receptors are used as a biomarker for urological diseases. The polypeptide sequences for interleukin receptors are known in the art. See for example, Genebank Accession Nos. NP000408 (IL-2 receptor α) and NP000869 (IL-2 receptor β).

C. Growth Factors

Another embodiment provides a method for diagnosing or assisting in the diagnosis of an urological disorder by determining the concentration of one or more growth factors in an urine sample of a subject and comparing the concentration of the one or more growth factors in the urine sample to the concentration of the one or more growth factors in the urine sample of a control, for example a subject that does not have an urological disorder. Concentrations of the one or more growth factors in urine that are higher than the control or reference level of the one or more growth factors are indicative of an urological disorder, for example overactive bladder syndrome. In the most preferred embodiment, the growth factor is epidermal growth factor (EGF).

A growth factor is a naturally occurring substance, usually a protein or a steroid, that is capable of stimulating cellular growth, proliferation and cellular differentiation. Growth factors are important for regulating a variety of cellular processes. Examples of growth factors include, but are not limited to, vascular endothelial growth factor (VEGF), bone morphogenetic protein (BMP), a transforming growth factor (TGF) such as transforming growth factor β, a platelet derived growth factor (PDGF), an epidermal growth factor (EGF), a nerve growth factor (NGF), an insulin-like growth factor (e.g., insulin-like growth factor I), scatter factor/hepatocyte growth factor (HGF), granulocyte/macrophage colony stimulating factor (GMCSF), a glial growth factor (GGF), and a fibroblast growth factor (FGF). The polypeptide sequences for growth factors are known in the art. See for example, Genebank Accession Nos. NP001954 (EGF precursor) and AAS83395 (EGF).

D. Normalizing Samples

Test and control samples can be normalized in one or more ways before the values are compared. Creatinine correction for urine levels of biomarkers is routinely used in urine proteomics and is also valid for studies involving measurement of urine chemokines believed to be passively released into urine from urothelium during the storage phase of voiding (Goto, et al., Allergol. Int, 56:433-438 (2007), Zimmerli, et al., Mol Cell Proteom., 7(2):290-298 (2008), Malayappan, et al., J. Chromatogr., 1167:54-62 (2007)). Since passive secretion of chemokines from the bladder to the stored urine is independent of the urine flow rate downstream of kidney, creatinine adjustment together with volume of urine voided at the time of collection is a suitable method for correcting the dilution status of urine. It has been reported that urinary creatinine concentration is influenced by age, sex, race/ethnicity, diet, and time of the day for spot urine collection (Boeniger, et al., Am. Ind. Hyg. Assoc. J., 54:615-627 (1993)). Variances resulting from the time of day for urine collection can be controlled by defining a standard window of time when each urine specimen is collected. The method of multiple regression can be used to adjust for variations in creatinine due to age and sex by weighted multivariate analysis of urinary creatinine. The bladder source of these biomarkers can be confirmed by their comparative detection in plasma or renal urine collected by ureteral catheters (Candela, et al., Urol. Res. 26:175-180 (1998)).

One method for diagnosing or assisting in the diagnosis of an urological disorder is by determining the concentration of a biomarker in an urine sample of a subject and comparing the concentration of the biomarker in the urine sample to the concentration of the biomarker in the urine sample of a control, after creatinine correction and multiple regression of the samples.

E. Reporting or Recording a Diagnosis

The disclosed methods of detecting or diagnosing an urological disorder based on concentrations of biomarkers in urine optionally include the step of reporting or recording the diagnosis of the urological disorder, which may include generating a report. The report can include the concentrations of biomarkers identified in the urine sample and likelihood the subject has an urological disorder. Reference levels for the biomarkers can also be included in the report. The report can be in any medium including, but not limited to, an oral report, a written report, or an electronic report which may, for example, be posted or access via website. Typically the report includes the concentrations of chemokines such as CCL2, CCL4 (MIP-1β), CCL11, and/or CXCL1 (GRO-α), cytokines such as sCD40L, IL-12p70/p40, IL-5, sIL-2Rα, IL-6, IL-10, and/or IL-8; growth factors such as epidermal growth factor (EGF), and/or combinations thereof in the urine sample, or the diagnosis resulting from an analysis of the levels. A preferred report includes the concentrations of CCL2, CCL4, CCL11 or combinations thereof.

The concentrations of biomarkers in the urine sample can serve as the basis for the diagnosis of an urological disorder such as interstitial cystitis/painful bladder syndrome, or overactive bladder syndrome. Alternatively, the results of one or more additional tests can be combined to arrive at the diagnosis. Other tests include, but are not limited to, one or more of the following: urine culture, cystoscopy, biopsy of the bladder wall, distention of the bladder under anesthesia, urine cytology, and, in men, laboratory examination of prostate secretions.

F. Discriminating Between Urological Disorders

The methods of detecting or diagnosing an urological disorder based on concentrations of biomarkers in urine can also be used to distinguish between two or more urological disorders. Urological disorders have unique biomarker profiles. Therefore, levels of biomarkers in a test subject can be compared to the reference levels of the same biomarkers in one or more subjects with a previously diagnosed urological disorder, to diagnose the urological disorder in the test subject. A method for discriminating between two urological disorders is by determining the concentration of one or more biomarkers in an urine sample of a subject with an undiagnosed urological disorder and comparing the concentration of the same one or more biomarkers in the urine sample to the concentration of the same one or more biomarkers in the urine sample of a reference subject, for example a subject that has been previously diagnosed with urological disorder. In some embodiments the “reference subject” is an average value from more than one subject previously diagnosed with the disorder. Relative concentrations of one or more biomarkers in urine that are similar to the level of the same one or more biomarkers in a subject with a known urological disorder, are indicative of the urological disorder. Biornarkers may be elevated, reduced, or unchanged compared to asymptomatic subjects.

In some cases one biomarker is used to discriminate between two or more diseases. In a preferred embodiment, more than one biomarker is used. When more than one biomarker is used, forward selection and Akaike information criterion for variable selection and model comparison by logistic regression are utilized to improve the diagnosis. In a preferred embodiment, levels of CCL2, IL-5 and eotaxin are analyzed to discriminate between IC/PBS and OAB, illustrated in Example 4.

Bacterial infection is reported to induce higher levels of chemokines from the CXC family vs. the CC family (Otto, et al., Kidney Int., 68:62-70 (2005)). Example 3 illustrates that similar levels of both CC chemokines and CXC chemokines in the urine of OAB patients without urinary tract infection (UTI) suggests that different inflammatory pathways are activated following UTI versus OAB. The quantitative differences in the elevation of specific CC chemokines and CXC chemokines in patients with UTI and OAB can therefore be used to discriminate between infectious and non-infectious bladder inflammation. In a preferred embodiment, levels of CCL2 and MIP-1β (CC chemokines) and CXCL1 and CXCL8 (CXCL chemokines) are analyzed to discriminate between infectious and non-infectious urological disorderssuch as urinary tract infection and overactive bladder syndrome.

### III. Methods for Detecting Chemokine Biomarkers Indicative of Urological Disorders

A. Immunoassays

The levels of biomarker protein can be measured by any means known to those skilled in the art. Preferred biomarker proteins include, but are not limited to, CCL2, CCL4 (MIP-1β), CCL11, CXCL1 (GRO-α), sCD40L, IL-12p70/p40, IL-5, sIL-2Rα, IL-6, IL-10, IL-8, and EGF. It is generally preferred to use antibodies, or antibody equivalents, to detect levels of biomarker protein. However, other methods for detection of biomarker expression can also be used. Biomarker protein activity can also be measured, for example, chemotactic activity of CCL2 can be measured.

In one embodiment, levels of biomarker protein are measured by contacting the biological sample with an antibody-based binding moiety that specifically binds to the biomarker protein, or to a fragment of the biomarker protein. Formation of the antibody-biomarker protein complex is then detected as a measure of biomarker protein levels.

The term “antibody-based binding moiety” or “antibody” includes immunoglobulin molecules and immunologically active determinants of immunoglobulin molecules, e.g., molecules that contain an antigen binding site which specifically binds (immunoreacts with) to the biomarker protein. The term “antibody-based binding moiety” is intended to include whole antibodies, e.g., of any isotype (IgG, IgA, IgM, IgE, etc.), and includes fragments thereof which are also specifically reactive with the biomarker protein. Antibodies can be fragmented using conventional techniques. Thus, the term includes segments of proteolytically-cleaved or recombinantly-prepared portions of an antibody molecule that are capable of selectively reacting with a certain protein. Non limiting examples of such proteolytic and/or recombinant fragments include Fab, F(ab′)2, Fab′, Fv, dAbs and single chain antibodies (scFv) containing a VL and VH domain joined by a peptide linker. The scFv's may be covalently or non-covalently linked to form antibodies having two or more binding sites. Thus, “antibody-based binding moiety” includes polyclonal, monoclonal, or other purified preparations of antibodies and recombinant antibodies. The term “antibody-based binding moiety” is further intended to include humanized antibodies, bispecific antibodies, and chimeric molecules having at least one antigen binding determinant derived from an antibody molecule. In a preferred embodiment, the antibody-based binding moiety detectably labeled.

The term “labeled antibody” includes antibodies that are labeled by a detectable means and include, but are not limited to, antibodies that are enzymatically, radioactively, fluorescently, and chemiluminescently labeled. Antibodies can also be labeled with a detectable tag, such as c-Myc, HA, VSV-G, HSV, FLAG, V5, or HIS.

In the diagnostic methods that use antibody based binding moieties for the detection of biomarker levels, the level of biomarker present in the biological samples correlate to the intensity of the signal emitted from the detectably labeled antibody.

In one preferred embodiment, the antibody-based binding moiety is detectably labeled by linking the antibody to an enzyme. The enzyme, in turn, when exposed to its substrate, will react with the substrate to produce a chemical moiety which can be detected, for example, by spectrophotometric, fluorometric or by visual means. Enzymes which can be used to detectably label the antibodies include, but are not limited to, malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-VI-phosphate dehydrogenase, glucoamylase and acetylcholinesterase. Chemiluminescence is another method that can be used to detect an antibody-based binding moiety.

Detection may also be accomplished using any of a variety of other immunoassays. For example, by radioactively labeling an antibody, it is possible to detect the antibody through the use of radioimmune assays. The radioactive isotope can be detected by such means as the use of a gamma counter or a scintillation counter or by autoradiography. Isotopes which are particularly useful are ³H, ¹³¹I, ³⁵S, ¹⁴C, and preferably ¹²⁵I.

It is also possible to label an antibody with a fluorescent compound. When the fluorescently labeled antibody is exposed to light of the proper wave length, its presence can then be detected due to fluorescence. Among the most commonly used fluorescent labeling compounds are CYE dyes, fluorescein isothiocyanate, rhodamine, phycoerytherin, phycocyanin, allophycocyanin, o-phthaldehyde and fluorescamine.

An antibody can also be detectably labeled using fluorescence emitting metals such as ¹⁵²Eu, or others of the lanthanide series. These metals can be attached to the antibody using such metal chelating groups as diethylenetriaminepentaacetic acid (DTPA) or ethylenediaminetetraacetic acid (EDTA).

An antibody also can be detectably labeled by coupling it to a chemiluminescent compound. The presence of the chemiluminescent-antibody is then determined by detecting the presence of luminescence that arises during the course of a chemical reaction. Examples of particularly useful chemiluminescent labeling compounds are luminol, luciferin, isoluminol, theromatic acridinium ester, imidazole, acridinium salt and oxalate ester.

In one preferred embodiment the biomarker proteins are detected by immunoassays, such as enzyme linked immunoabsorbant assay (ELISA), radioimmunoassay (RIA), immunoradiometric assay (IRMA), or Western blotting. These assays are well known to those skilled in the art. Immunoassays such as ELISA or RIA, which can be extremely rapid, are more generally preferred. Antibody arrays or protein chips can also be employed, see for example U.S. Pat. Nos. 6,329,209 and 6,365,418.

The most common enzyme immunoassay is the “Enzyme-Linked Immunosorbent Assay (ELISA).” ELISA is a technique for detecting and measuring the concentration of an antigen using a labeled (e.g., enzyme linked) form of the antibody. There are different forms of ELISA, which are well known to those skilled in the art. The standard techniques known in the art for ELISA are described in “Methods in Immunodiagnosis”, 2nd Edition, Rose and Bigazzi, eds. John Wiley & Sons, 1980; Campbell et al., “Methods and Immunology”, W. A. Benjamin, Inc., 1964; and Oellerich, M., J. Clin. Chem. Clin. Biochem., 22:895-904 (1984).

In a “sandwich ELISA”, an antibody specific for the biomarker protein is linked to a solid phase (i.e., a microtiter plate) and exposed to a biological sample containing antigen (e.g., biomarker protein). The solid phase is then washed to remove unbound antigen. A labeled antibody (e.g., enzyme linked) is then bound to the bound-antigen (if present) forming an antibody-antigen-antibody sandwich. Examples of enzymes that can be linked to the antibody are alkaline phosphatase, horseradish peroxidase, luciferase, urease, and B-galactosidase. The enzyme linked antibody reacts with a substrate to generate a colored reaction product that can be measured.

In a “competitive ELISA”, antibody is incubated with a sample containing the biomarker protein (i.e., antigen). The antigen-antibody mixture is then contacted with a solid phase (e.g., a microtiter plate) that is coated with antigen. The more antigen present in the sample, the less free antibody that will be available to bind to the solid phase. A labeled (e.g., enzyme linked) secondary antibody is then added to the solid phase to determine the amount of primary antibody bound to the solid phase.

A preferred detect assay is the Luminex® assay, as described in the Examples below. Cytokines, chemokines, and growth factors can be assayed using commercially available microspheres (Millipore, Billerica, Mass.). Different from conventional ELISA, in the multiplex Luminex® assay format, multiplex capture antibody is attached to a polystyrene bead rather than attached to the microplate well. Beads covalently bound to different antibodies can be mixed in the same assay, utilizing a 96-well microplate format. For example, microspheres of defined spectral properties conjugated to antibodies directed against urinary proteins can be brought into contact with a sample, such as urine. After the sandwich immunoassay assay is complete, beads can be read, using the Luminex® 100™ or 200™ detection system.

Other techniques may be used to detect the biomarkers, according to a practitioner's preference, and based upon the present disclosure. One such technique is Western blotting (Towbin et al., Proc. Nat. Acad. Sci. 76:4350 (1979)), wherein a suitably treated sample is run on an SDS-PAGE gel before being transferred to a solid support, such as a nitrocellulose filter. Detectably labeled antibodies that specifically bind to biomarker proteins can then be used to assess biomarker levels, where the intensity of the signal from the detectable label corresponds to the amount of biomarker present. Levels can be quantitated, for example by densitometry.

B. Mass spectrometry

Biomarkers for urological disorders may be detected using mass spectrometry such as MALDI/TOF (time-of-flight), SELDI/TOF, liquid chromatography-mass spectrometry (LC-MS), gas chromatography-mass spectrometry (GC-MS), high performance liquid chromatography-mass spectrometry (HPLC-MS), capillary electrophoresis-mass spectrometry, nuclear magnetic resonance spectrometry, or tandem mass spectrometry (e.g., MS/MS, MS/MS/MS, ESI-MS/MS, etc.).

Mass spectrometry methods are well known in the art and have been used to quantify and/or identify biomolecules, such as proteins (see, e.g., Chen, C. H., Anal Chim Acta, 624(1):16-36 (2008)). Further, mass spectrometric techniques have been developed that permit at least partial de novo sequencing of isolated proteins.

In certain embodiments, a gas phase ion spectrophotometer is used. In other embodiments, laser-desorption/ionization mass spectrometry is used to analyze the sample. Modern laser desorption/ionization mass spectrometry (“LDI-MS”) can be practiced in two main variations: matrix assisted laser desorption/ionization (“MALDI”) mass spectrometry and surface-enhanced laser desorption/ionization (“SELDI”). In MALDI, the analyte is mixed with a solution containing a matrix, and a drop of the liquid is placed on the surface of a substrate. The matrix solution then co-crystallizes with the biological molecules. The substrate is inserted into the mass spectrometer. Laser energy is directed to the substrate surface where it desorbs and ionizes the biological molecules without significantly fragmenting them. However, MALDI has limitations as an analytical tool. It does not provide means for fractionating the sample, and the matrix material can interfere with detection, especially for low molecular weight analytes.

In SELDI, the substrate surface is modified so that it is an active participant in the desorption process. In one variant, the surface is derivatized with adsorbent and/or capture reagents that selectively bind the protein of interest. In another variant, the surface is derivatized with energy absorbing molecules that are not desorbed when struck with the laser. In another variant, the surface is derivatized with molecules that bind the protein of interest and that contain a photolytic bond that is broken upon application of the laser. In each of these methods, the derivatizing agent generally is localized to a specific location on the substrate surface where the sample is applied. The two methods can be combined by, for example, using a SELDI affinity surface to capture an analyte and adding matrix-containing liquid to the captured analyte to provide the energy absorbing material.

Detection of the presence of a marker or other substances will typically involve detection of signal intensity. This, in turn, can reflect the quantity and character of a polypeptide bound to the substrate. For example, in certain embodiments, the signal strength of peak values from spectra of a first sample and a second sample can be compared (e.g., visually, by computer analysis etc.), to determine the relative amounts of particular biomolecules. Software programs such as the Biomarker Wizard program (Ciphergen Biosystems, Inc., Fremont, Calif.) can be used to aid in analyzing mass spectra. The mass spectrometers and their techniques are well known to those of skill in the art.

Any person skilled in the art understands, any of the components of a mass spectrometer (e.g., desorption source, mass analyzer, detect, etc.) and varied sample preparations can be combined with other suitable components or preparations described herein, or to those known in the art. For example, in some embodiments a control sample may contain heavy atoms (e.g., ¹³C) thereby permitting the test sample to mixed with the known control sample in the same mass spectrometry run.

In one preferred embodiment, a laser desorption time-of-flight (TOF) mass spectrometer is used. In laser desorption mass spectrometry, a substrate with a bound marker is introduced into an inlet system. The marker is desorbed and ionized into the gas phase by laser from the ionization source. The ions generated are collected by an ion optic assembly, and then in a time-of-flight mass analyzer, ions are accelerated through a short high voltage field and let drift into a high vacuum chamber. At the far end of the high vacuum chamber, the accelerated ions strike a sensitive detector surface at a different time. Since the time-of-flight is a function of the mass of the ions, the elapsed time between ion formation and ion detector impact can be used to identify the presence or absence of molecules of specific mass to charge ratio.

In some embodiments the relative amounts of one or more biomarkers present in a first or second sample is determined, in part, by executing an algorithm with a programmable digital computer. The algorithm identifies at least one peak value in the first mass spectrum and the second mass spectrum. The algorithm then compares the signal strength of the peak value of the first mass spectrum to the signal strength of the peak value of the second mass spectrum of the mass spectrum. The relative signal strengths are an indication of the amount of the biomarker that is present in the first and second samples. A standard containing a known amount of a biomarker can be analyzed as the second sample to provide better quantify the amount of the biomarker present in the first sample. In certain embodiments, the identity of the biomolecules in the first and second sample can also be determined.

In one preferred embodiment, biomarker levels are measured by MALDI-TOF mass spectrometry.

C. Methods for Assessing the Severity of Urological Disorders

Concentrations of biomarkers in urine samples of as subject can also be used to assess the severity of any urological disorder the subject may have. Generally, the higher the concentration of biomarkers such as CCL2, CCL4 (MIP-1β), CCL11, CXCL1 (GRO-α), sCD40L, IL-12p70/p40, IL-5, sIL-2Rα, IL-6, IL-10, IL-8, EGF or combinations thereof relative to a control, the more severe the urological condition. Reference levels of biomarkers in urine samples from healthy subject without urological disorders or symptoms thereof can be used to establish a reference level or concentration of biomarkers.

One method for assessing the severity of an urological disorder includes assaying an urine sample for concentrations of biomarkers, wherein the concentrations of the biomarkers are correlated to the severity of an urological disorder. The method optionally includes the step of reporting or recording the severity of the urological disorder based on the concentrations of the biomarkers in the urine sample. The concentration of CCL2, CCL4 (MIP-1β), CCL11, CXCL1 (GRO-α), sCD40L, IL-12p70/p40, IL-5, sIL-2Rα, IL-6, IL-10, IL-8, EGF and/or combinations thereof can be monitored over the course of treatment. Preferred biomarkers for assessing the severity of IC/PBS are chemokines relevant to chemotaxis of eosinophils, chemotaxis of monocytes, and activation of mast cells. Most preferred biomarkers are CCL2, CCL4, CCL11, and IL-8. The course of treatment can take place over hours, days, weeks, or months.

Another embodiment provides a method for assessing the effectiveness of a treatment for an urological disorder by assaying an urine sample for concentrations of biomarkers from a subject undergoing treatment for the urological disorder, wherein elevated concentrations of the biomarkers in the urine sample from the subject indicates the treatment for the urological disorder is ineffective. The method optionally includes the step of recording or reporting the effectiveness of the treatment based on the concentrations of the biomarkers in the urine sample.

Although in the preferred embodiment, these methods and reagents are used to provide reports as to the existence and severity, if any, of a disease or disorder, kits may also be provided which contain the reagents for testing samples for the presence and amount of biomarkers associated with diseases or disorders for which comparative values are known. The kits may also provide software and/or positive and negative controls for use in testing of samples, and determining the presence of disease or disorder, or the risk thereof.

### IV. Methods of Treating Urological Disorders

Methods for treating urological disorders, in particular interstitial cystitis/painful bladder syndrome, are provided. In one embodiment, the biological activity of biomarkers in the bladder is inhibited or reduced relative to a control. In another embodiment, the expression of chemokines in the bladder is inhibited or reduced relative to a control.

A. Inhibiting Cytokine and Chemokine Activity

1. Antibodies

Reducing the biological activity of chemokines and cytokines relevant to urological disorders is effective to treat certain urological disorders, in particular interstitial cystitis/painful bladder syndrome and overactive bladder syndrome. In a preferred method for treating interstitial cystitis/painful bladder syndrome the biological activity of chemokines relevent to chemotaxis of eosinophils, chemotaxis of monocytes, and activation of mast cells in the bladder of a subject are reduced. Chemokines targeted for inhibition include, but are not limited to, one or more of CCL2, CCL4 (MIP-1β), CCL11, CXCL1 (GRO-α), sCD40L, IL-12p70/p40, IL-5, sIL-2Rα, IL-6, IL-10, and IL-8.

An effective amount of a chemokine or cytokine antagonist to diminish the severity or number of symptoms of an urological disorder is administered to a subject having one or more symptoms of an urological disorder. As used herein, the terms “inhibitors” or “antagonists” refers to compounds or compositions that directly or indirectly partially or totally block activity, decrease, prevent, delay activation, inactivate, desensitize, or down regulate the activity or expression of the targeted chemokine biomarker. Antagonists can be, for example, polypeptides such as antibodies and soluble receptors, as well as nucleic acids such as siRNA or antisense RNA, as well as naturally occurring or synthetic biomarker antagonists, including small chemical molecules.

A preferred chemokine or cytokine antagonist is an antibody or antigen-binding fragment thereof that binds to the chemokine or cytokine and prevents the chemokine or cytokine from binding to receptors for the chemokine or cytokine. Suitable antibody fragments include Fab, F(ab′)2, Fab′, Fv, dAbs.

The antibodies can be polyclonal, monoclonal, humanized or single chain antibodies (scFv) containing a VL and VH domain joined by a peptide linker. Antibodies to CCL2, CCL4 (MIP-1β), CCL11, CXCL1 (GRO-α), sCD40L, IL-12p70/p40, IL-5, sIL-2Rα, IL-6, IL-10, and IL-8 are commercially available.

Another embodiment provides administering an effective amount of a chemokine or cytokine antagonist to subject in need thereof, wherein the chemokine or cytokine antagonist includes an antibody that binds to the receptor for the chemokine or cytokine and inhibits binding of the chemokine or cytokine to the receptor relative to a control.

2. Small Molecule Chemokine and Cytokine Antagonists

Chemokine and cytokine antagonists also include small molecule antagonist molecules. Small molecule chemokine or cytokine antagonists are typically less than 1,000 daltons, more typically about 500 daltons, and are carbon-based molecules. Small molecule chemokine or cytokine antagonists can be identified by screening combinatorial libraries for molecules that binding the chemokine or cytokine or the receptor for the chemokine or cytokine and inhibit or reduce signal transduction through the receptor.

B. Down-Regulating Expression of Chemokines and Cytokines

In another embodiment, antagonists reduce or inhibit biomarkers relevant to urological disorders in a subject. Preferred biomarkers include, but are not limited to, CCL2, CCL4 (MIP-1β), CCL11, CXCL1 (GRO-α), sCD40L, IL-12p70/p40, IL-5, sIL-2Rα, IL-6, IL-10, and IL-8. In a preferred embodiment, antagonists reduce or inhibit the expression of chemokines relevant to chemotaxis of eosinophils, chemotaxis of monocytes, and activation of mast cells in the bladder of a subject with IC/PBS. Preferred chemokines to be down-regulated include CCL2, CCL4, and CCL11 and combinations thereof. Antagonists that reduce or inhibit expression of these chemokines or cytokines include inhibitory nucleic acids, including, but not limited to, ribozymes, triplex-forming oligonucleotides (TFOs), antisense DNA, siRNA, and microRNA specific for nucleic acids encoding the chemokines. The antisense DNA oligonucleotides typically include at least 18, 19, 20, 21, 22, 23, 24 or 25 nucleotides and are preferably at least 20 nucleotides in length.

Useful inhibitory nucleic acids include those that reduce the expression of RNA encoding chemokines or cytokines in the bladder by at least 20%, 30%, 40%, 50%, 60%, 70%, 80%, 90% or 95% compared to controls. Expression of chemokines or cytokines in the bladder can be measured by methods well known to those of skill in the art, including northern blotting and quantitative polymerase chain reaction (PCR).

Inhibitory nucleic acids and methods of producing them are well known in the art. siRNA design software is available, for example, at http://i.cs.hku.hk/˜-sirna/software/sirna.php. Synthesis of nucleic acids is well known, see, for example, Molecular Cloning: A Laboratory Manual (Sambrook and Russel eds. 3^{rd} ed.) Cold Spring Harbor, N.Y. (2001). The term “siRNA” means a small interfering RNA that is a short-length double-stranded RNA that is not toxic. Generally, there is no particular limitation of the length of siRNA as long as it does not show toxicity. “siRNAs” can be, for example, 15 to 49 bp, preferably 15 to 35 bp, and more preferably 21 to 30 by long. Alternatively, the double-stranded RNA portion of a final transcription product of siRNA to be expressed can be, for example, 15 to 49 bp, preferably 15 to 35 bp, and more preferably 21 to 30 by long. In a preferred embodiment, the siRNA is at least 19, 20, 21, 22, or 23 nucleotides long. The double-stranded RNA portions of siRNAs in which two RNA strands pair up are not limited to the completely paired ones, and may contain nonpairing portions due to mismatch (the corresponding nucleotides are not complementary), or bulge (lacking in the corresponding complementary nucleotide on one strand). Non-pairing portions can be contained to the extent that they do not interfere with siRNA formation. The “bulge” used herein preferably comprise 1 to 2 nonpairing nucleotides, and the double-stranded RNA region of siRNAs in which two RNA strands pair up contains preferably 1 to 7, more preferably 1 to 5 bulges. In addition, the “mismatch” used herein is contained in the double-stranded RNA region of siRNAs in which two RNA strands pair up, preferably 1 to 7, more preferably 1 to 5, in number. In a preferable mismatch, one of the nucleotides is guanine, and the other is uracil. Such a mismatch is due to a mutation from C to T, G to A, or mixtures thereof in DNA coding for sense RNA, but not particularly limited to them. Furthermore, the double-stranded RNA region of siRNAs in which two RNA strands pair up may contain both bulge and mismatched, which sum up to, preferably 1 to 7, more preferably 1 to 5 in number.

The terminal structure of siRNA may be either blunt or cohesive (overhanging) as long as siRNA can silence, reduce, or inhibit the target gene expression due to its RNAi effect. The cohesive (overhanging) end structure is not limited only to the 3′ overhang, and the 5′ overhanging structure may be included as long as it is capable of inducing the RNAi effect. In addition, the number of overhanging nucleotide is not limited to the already reported 2 or 3, but can be any numbers as long as the overhang is capable of inducing the RNAi effect. For example, the overhang consists of 1 to 8, preferably 2 to 4 nucleotides. Herein, the total length of siRNA having cohesive end structure is expressed as the sum of the length of the paired double-stranded portion and that of a pair comprising overhanging single-strands at both ends. For example, in the case of 19 by double-stranded RNA portion with 4 nucleotide overhangs at both ends, the total length is expressed as 23 bp. Furthermore, since this overhanging sequence has low specificity to a target gene, it is not necessarily complementary (antisense) or identical (sense) to the target gene sequence. Furthermore, as long as siRNA is able to maintain its gene silencing effect on the target gene, siRNA may contain a low molecular weight RNA (which may be a natural RNA molecule such as tRNA, rRNA or viral RNA, or an artificial RNA molecule), for example, in the overhanging portion at its one end.

In addition, the terminal structure of the siRNA is not necessarily the cut off structure at both ends as described above, and may have a stern-loop structure in which ends of one side of double-stranded RNA are connected by a linker RNA. The length of the double-stranded RNA region (stem-loop portion) can be, for example, 15 to 49 bp, preferably 15 to 35 bp, and more preferably 21 to 30 by long. Alternatively, the length of the double-stranded RNA region that is a final transcription product of siRNAs to be expressed is, for example, 15 to 49 bp, preferably 15 to 35 bp, and more preferably 21 to 30 by long. Furthermore, there is no particular limitation in the length of the linker as long as it has a length so as not to hinder the pairing of the stem portion. For example, for stable pairing of the stem portion and suppression of the recombination between DNAs coding for the portion, the linker portion may have a clover-leaf tRNA structure. Even though the linker has a length that hinders pairing of the stem portion, it is possible, for example, to construct the linker portion to include introns so that the introns are excised during processing of precursor RNA into mature RNA, thereby allowing pairing of the stem portion. In the case of a stem-loop siRNA, either end (head or tail) of RNA with no loop structure may have a low molecular weight RNA. As described above, this low molecular weight RNA may be a natural RNA molecule such as tRNA, rRNA or viral RNA, or an artificial RNA molecule.

miRNAs are produced by the cleavage of short stem-loop precursors by Dicer-like enzymes; whereas, siRNAs are produced by the cleavage of long double-stranded RNA molecules. miRNAs are single-stranded, whereas siRNAs are double-stranded.

Methods for producing miRNA are known in the art. Because the sequences for CCL2, CCL4 (MIP-1β), CCL11, CXCL1 (GRO-α), sCD40L, IL-12p70/p40, IL-5, sIL-2Rα, IL-6, IL-10, IL-8, and EGF are known, one of skill in the art could readily produce miRNAs that downregulate expression of these chemokines using information that is publicly available.

C. Administration of Growth Factors

Increasing the biological activity of growth factors relevant to urological disorders is effective to treat certain urological disorders, in particular interstitial cystitis/painful bladder syndrome and overactive bladder syndrome. The presence of elevated levels of EGF in urine of patients with overactive bladder syndrome is suggestive of tissue repair and fibrosis. An effective amount of one or more growth factors to diminish the severity or number of symptoms of an urological disorder is administered to a subject having one or more symptoms of an urological disorder. The preferred growth factor is epidermal growth factor (EGF).

D. Methods of Administration

The chemokine or cytokine antagonist, or growth factor can be formulated into a pharmaceutical composition, and is preferably administered by instillation into the bladder, most preferably using liposomal carriers. Methods of instillation are known. (Lawrencia, et al., Gene Ther, 8:760-8 (2001); Nogawa, et al., J Clin Invest, 115:978-85 (2005); Ng, et al., Methods Enzymol, 391:304-13 (2005); Tyagi, et al., J Urol, 171:483-9 (2004). (Trevisani, et al., J Pharmacol Exp Ther, 309:1167-73 (2004); Trevisani, et al., Nat Neurosci, 5:546-51 (2002)).

The selected dosage depends upon the desired therapeutic effect, on the route of administration, and on the duration of the treatment desired. Generally, for intravenous injection or infusion, dosage may be lower. As used herein the term “effective amount” or “therapeutically effective amount” means a dosage sufficient to treat, inhibit, or alleviate one or more symptoms of the disorder or disease being treated or to otherwise provide a desired pharmacologic and/or physiologic effect. The precise dosage will vary according to a variety of factors such as subject-dependent variables (e.g., age, immune system health, etc.), the disease, and the treatment being effected.

E. Pharmaceutical Formulations

In general, pharmaceutical compositions are provided including effective amounts of one or more chemokine or cytokine antagonist, or growth factor and optionally include pharmaceutically acceptable diluents, preservatives, solubilizers, emulsifiers, adjuvants and/or carriers. Such compositions include diluents sterile water, buffered saline of various buffer content (e.g., Tris-HCl, acetate, phosphate), pH and ionic strength; and optionally, additives such as detergents and solubilizing agents (e.g., TWEEN® 20, TWEEN® 80, Polysorbate 80), anti-oxidants (e.g., ascorbic acid, sodium metabisulfite), and preservatives (e.g., Thimersol, benzyl alcohol) and bulking substances (e.g., lactose, mannitol). Examples of non-aqueous solvents or vehicles are propylene glycol, polyethylene glycol, vegetable oils, such as olive oil and corn oil, gelatin, and injectable organic esters such as ethyl oleate. The formulations may be lyophilized and re-dissolved/resuspended immediately before use. The formulation may be sterilized by, for example, filtration through a bacteria retaining filter, by incorporating sterilizing agents into the compositions, by irradiating the compositions, or by heating the compositions.

Liposomes are lipid vesicles composed of concentric phospholipid bilayers which enclose an aqueous interior (Gregoriadis, et al., Int J Pharm 300, 125-30 2005; Gregoriadis and Ryman, Biochem J 124, 58P (1971)). The lipid vesicles comprise either one or several aqueous compartments delineated by either one (unilamellar) or several (multilamellar) phospholipid bilayers (Sapra, et al., Curr Drug Deliv 2, 369-81 (2005)). The success of liposomes in the clinic has been attributed to the nontoxic nature of the lipids used in their formulation. Both the lipid bilayer and the aqueous interior core of liposomes can serve the purpose of treatment. Liposomes have been well studied as carrier of toxins for enhancing their efficacy at lower doses (Alam, et al., Mol Cell Biochem 112, 97-107 1992; Chaim-Matyas, et al., Biotechnol Appl Biochem 17 (Pt 1), 31-6 1993; de Paiva and Dolly, FEBS Lett 277, 171-4 (1990); Freitas and Frezard, Toxicon 35, 91-100 (1997); Mandal and Lee, Bioehim Biophys Acta 1563, 7-17 (2002)).

Liposomes have the ability to form a molecular film on cell and tissue surfaces and are currently being tested as possible therapeutic agents to promote wound healing and healing dry eye as a tear substitute. Clinical studies have proven the efficacy of liposomes as a topical healing agent (Dausch, et al., Klin Monatsbl Augenheilkd 223, 974-83 (2006); Lee, et al., Klin Monatsbl Augenheilkd 221, 825-36 (2004)). Liposomes have also been used in opthalmology to ameliorate keratitis, corneal transplant rejection, uveitis, endophthalmitis, and proliferative vitreoretinopathy (Ebrahim, et al., 2005; Li, et al., 2007).

Liposomes have been widely studied as drug carriers for a variety of chemotherapeutic agents (approximately 25,000 scientific articles have been published on the subject) (Gregoriadis, N Engl J Med 295, 765-70 (1976); Gregoriadis, et al., Int. J. Pharm. 300, 125-30 (2005)). Water-soluble anticancer substances such as doxorubicin can be protected inside the aqueous compartment(s) of liposomes delimited by the phospholipid bilayer(s), whereas fat-soluble substances such as amphotericin and capsaicin can be integrated into the phospholipid bilayer (Aboul-Fadi, Curr Med Chem 12, 2193-214 (2005); Tyagi, et al., J Urol 171, 483-9 (2004)). Topical and vitreous delivery of cyclosporine was drastically improved with liposomes (Lallemand, et al., Eur Pharm Biopharm 56, 307-18 2003). Delivery of chemotherapeutic agents lead to improved pharmacokinetics and reduced toxicity profile (Gregoriadis, Trends Biotechnol 13, 527-37 (1995); Gregoriadis and Allison, FEBS Lett 45, 71-4 1974; Sapra, et al., Curr Drug Deliv 2, 369-81 (2005)). More than ten liposomal and lipid-based formulations have been approved by regulatory authorities and many liposomal drugs are in preclinical development or in clinical trials (Barnes, Expert Opin Pharmacother 7, 607-15 (2006); Minko, et al., Anticancer Agents Med Chem 6, 537-52 (2006)). The safety data with respect to acute, subchronic, and chronic toxicity of liposomes has been assimilated from the vast clinical experience of using liposomes in the clinic for thousands of patients.

### IV. Detection Kits

Kits for the detection and prognostic evaluation of urological disorders, including IC/PBS and OAB can be in any configuration well known to those of ordinary skill in the art which is useful for performing one or more of the methods described herein for the detection of biomarker proteins. The kits can supply many or all of the essential reagents for conducting an assay for the detection of biomarker protein in an urine sample. In addition, the assay is preferably performed simultaneously with a standard or multiple standards that are included in the kit, such as a predetermined amount of biomarker protein, so that the results of the test can be quantitated or validated. Kits also include reference samples and/or calculations for average values of biomarkers assayed in asymptomatic patients, normal individuals and/or patients with a known urological disorder.

The kits include an assay means for detecting biomarker levels such as antibodies, or antibody fragments, which selectively bind to biomarker protein. In one embodiment, the kits provide at least one antibody-based binding moiety that binds to at least one biomarker protein, e.g., CCL2, CCL4 (MIP-1β), CCL11, CXCL1 (GRO-α), sCD40L, IL-12p70/p40, IL-5, sIL-2Rα, IL-6, IL-10, IL-8, or EGF and a suitable container means. In certain embodiments, the kit may further include a second antibody preparation (preferably detectably labeled) that binds immunologically to the same biomarker protein as the first antibody preparation, but where the first and the second antibodies bind to different epitopes, and a suitable container means thereof. In one embodiment, the first antibody preparation is attached to a support. The support can be any support routinely used in immunological techniques. In a particularly preferred embodiments, the support independently is a polystyrene plate, test tube or dipstick.

The kits may include multiple antibodies that interact with each of the disclosed biomarker proteins, such that multiple biomarker proteins can be measured.

In other embodiments, the assay kits employ the following techniques to measure the level of biomarker protein: competitive and non-competitive assays, radioimmunoassay (RIA), bioluminescence and chemiluminescence assays, fluorometric assays, sandwich assays, immunoradiometric assays, dot blots, enzyme linked assays including ELISA, and immunocytochemistry. For each kit the range, sensitivity, precision, reliability, specificity and reproducibility of the assay are established by means well known to those skilled in the art.

It will be appreciated that the kit components are packaged in a container for sale and distribution.

### EXAMPLES

The present invention will be further understood by reference to the following non-limiting examples.

### Example 1

### Chemokines are Elevated in the Urine of Patients with Interstitial Cystitis/Painful Bladder Syndrome (IC/PBS)

Samples and Subjects

A total of 27 urine samples were collected from 8 healthy subjects and IC/PBS patients of different severity, 12 with mild to moderate IC/PBS and 7 with severe IC/PBS. Urinalysis was performed by dipstick, and no pyuria or bacteria suggestive of urinary tract infection was identified. In addition, urine cultures were performed on all samples from the subjects with IC/PBS, and all were sterile. The mean age was 51 years in the IC/PBS group, and 34 years in the control group. The number of years with disease was 6.4. The control group included individuals with no active urologic disease or symptoms at the time of urine collection or patients with stress urinary incontinence. The diagnosis of IC/PBS was based strictly on the National Institute of Arthritis, Diabetes, Digestive and Kidney Diseases criteria, and no subjects had had an urinary tract infection, hydrodistension, or any intravesical treatment for at least 12 weeks before the urine collection. Fresh voided urine was immediately centrifuged, aliquoted, and frozen in liquid nitrogen. Subjects completed the validated University of Wisconsin IC questionnaire at the time of urine collection.

Assay of Chemokines

An automated immunoassay analyzer (Luminex® 100™ IS System, Luminex, Austin, Tex.; purchased through MiraiBio, Alameda, Calif.) was used to assay for chemokines. The automated immunoassay analyzer is a continuous; random-access instrument that performs automated chemiluminescent immunoassays. Chemokines were assayed using commercially available microspheres (Milipore, Billerica, Mass.). The antibody-conjugated microspheres were allowed to react with urine sample and a secondary, or detection, antibody in a microplate well to form a capture sandwich immunoassay. The protocol for these assays involves mixing with antibody-coated microspheres, incubated in the dark at room temperature for overnight, washing, addition of detection antibody, addition of 25 μl Streptavidin-Phycoerythrin solution, incubation in the dark for 30 min (room temperature), washing three times, and reading on the Luminex® system.

Microspheres of defined spectral properties conjugated to antibody directed against specific chemokine were pipetted into the wells of a filter bottom microplate. A 96-well 1.2 μm filter plate was blocked for 2 min with 100 μl of PBS (pH7.4) with 1% bovine serum albumin and 0.02% sodium azide (PBN) and then washed once with 190 μl of PBS (pH7.4) with 0.05% Tween® 20 in PBS. Wells were kept moist by addition of 20 μl of PBS buffer. Urine (50 μl, diluted 1:100 in PBS+sodium azide buffer) were added to each well of the filter plate. Approximately 2,500 antibody-conjugated microspheres per protein were added to each well in 50 μl of PBN buffer. The plate were incubated on a shaker in the dark overnight at 25° C. and then washed three times with PBS-T using a vacuum manifold. Affinity-purified biotin-labeled goat anti-human IgG (50 μl of a 1:1000 dilution in PBN) were added, and the plate was incubated on a shaker in the dark for 30 min at 37° C. and then washed twice with PBS-T. Streptavidin-conjugated with R-phycoerythrin (50 μl of a 1:100 dilution in PBN buffer) was added, and the plate was incubated on a shaker in the dark for 30 min at 37° C. and then washed twice with PBS-T using a vacuum manifold. The microspheres were then resuspended in 125 μl of PBN per well, and 75 μl of suspension was transferred to clear the polystyrene 96-well plate. Microspheres were aspirated through the flow cell of a dual-laser Luminex® 100™ instrument. The median fluorescence intensity of 100 microspheres of each specific protein was recorded for each well.

Measurement of Creatinine

To correct for hydration status, chemokine concentrations were expressed as picograms per milligram creatinine. Filtered urine samples were injected in Waters HPLC instrument equipped with degasser, binary pump, and automatic sampler. An analytical column, Hypersil gold C₁₈ (150 mm 3.9 mm; Thermo fisher), was used at flow rate of 1.0 mL min⁻¹. The UV absorbance detector was fixed at 250 nm for creatinine. The mobile phase is formed by a mixture of citric acid buffer, pH 6.0, and acetonitrile with final mobile phase composition of 97% buffer solution and 3% acetonitrile to determine urinary creatinine. Quantification is based on peak height.

Statistical Analysis

Assay results are expressed as means±SE. Statistical differences of urinary chemokines between groups were determined by single factor ANOVA followed by the parametric Tukey test.

Results

Analysis of urine from single void of patients with IC/PBS revealed at least 10 fold elevation in the urine levels of CCL2 (p<0.05), MIP-1β (p<0.01) and eotaxin (p<0.01) compared to controls. The chemokine profile associated with IC/PBS has direct effects on the migration of eosinophils and monocytes in the presence of mast cells. These results show that an elevation of chemokines is linked to inflammatory cells previously studied in IC/PBS.

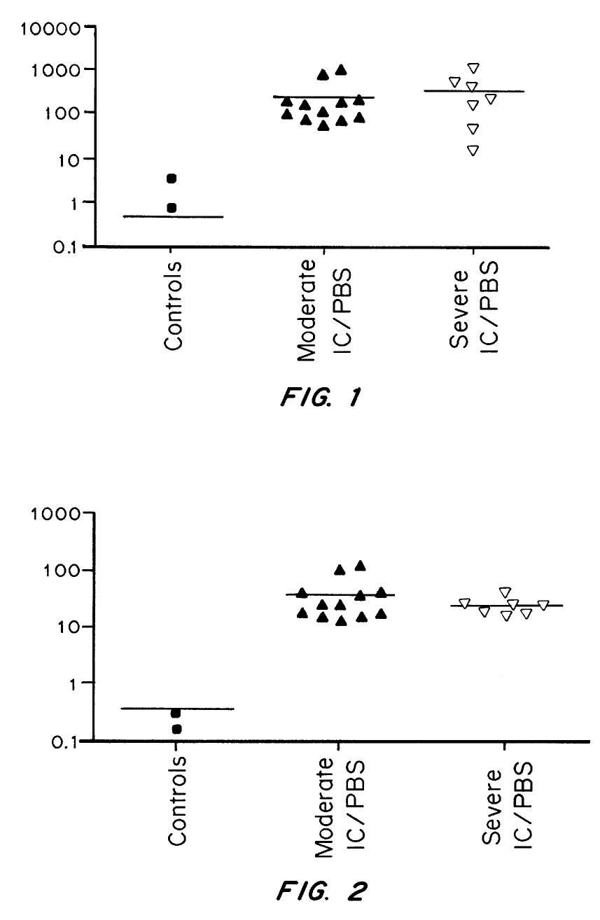

As the data shown in FIG. 1, the levels of CCL2 in urine of IC/PBS patients were 100 fold higher than levels measured in control group. The high levels of CCL2 are in agreement with increased presence of mast cells and macrophages in the detrusor and urothelium of IC/PBS patients (Koskela, L. R., et al., J Urol, 180:737 (2008)); Theoharides, T. C., et al. Mast cell involvement in interstitial cystitis: a review of human and experimental evidence. Urology, 57: 47, 2001. Levels of CCL2 were variable in patients with severe disease. The apparent paradoxical results of lower chemokine levels in severe patients compared to patients with mild to moderate disease condition can be explained by the drastically reduced bladder capacity of patients with the severe condition. Since chemokines in these patients are in reduced volumes of urine for reduced time periods for diffusion into from the bladder tissue, the chemokines concentrations translate into lower levels in single voids.

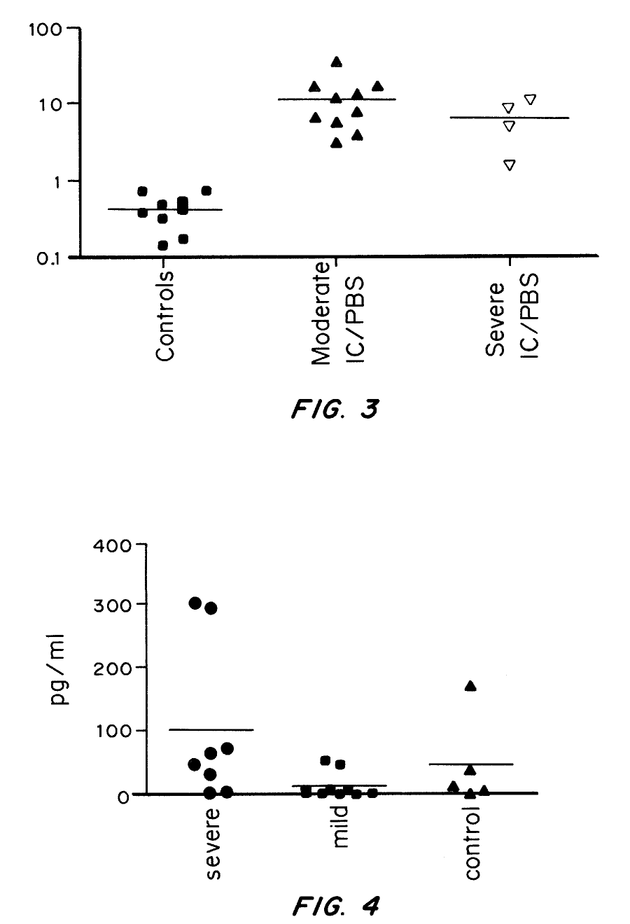

In contrast to CCL2 levels, the levels MIP-1β were uniformly 100 fold higher than levels measured in the control group (FIG. 2) (p<0.01). Elevated plasma levels of MIP-1β agree with chronic nature of the disease in patients with increased macrophage activity and probably associated with fibrosis as in lungs. Levels of Eotaxin were only 10 fold higher than the levels measured in control group (FIG. 3) (p<0.01). High levels of eotaxin agree with infiltration of eosinophils in tissue biopsy of patients. The levels of MIP-1β and eotaxin in patients with the severe condition were not significantly higher than control group to suggest that these chemokines are not good indicator of disease severity as CCL2.

### Example 2

### Chemokines can be Used to Objectively Grade Severity and Disease Progression in IC/PBS

Materials and Methods

Mid stream urine specimens from 17 IC/PBS patients with varying disease severity and their age, race, and sex matched 5 asymptomatic control subjects were analyzed using multiplex immunoassays based on 8-panel Luminex® xMAP kit available from Millipore. The chemokine concentrations expressed as pg/ml were analyzed by Kruskal analyses of variance followed by Mann-Whitney U-test.

Results

Interstitial cystitis/painful bladder syndrome (IC/PBS) is a difficult disease to diagnose and treat. Development of objective biomarkers for diagnosis and grading of severity and disease progression is therefore a high priority.

Urinalysis of mild to moderate and severe IC/PBS patients revealed elevation in the levels of 8 chemokines relative to controls in both disease categories. However, severity of disease condition in IC/PBS patients was associated with significant elevation of IL-8 in the urine of these patients compared to patients of mild condition (p<0.05). Elevation of IL-8 ensures infiltration of neutrophils in bladder to sustain inflammation. The absence of pyuria in these IC/PBS patients can be explained by elevation of sIL-1RA in both patients and controls, which counters the urinary tract infection by blocking the deleterious effects of IL-1a in bladder.

IL-8 is secreted by macrophages and urothelium and its elevation in severe IC/PBS compared to patients with mild condition is consistent with severity of disease condition presumably due to severe inflammation. Therefore it can be inferred from this data that urinary chemokines reflect the changes in paracrine signaling within the diseased bladder and this knowledge can be used to guide therapy and improved management of this complex disease. The chemokines repertoire of bladder can be used to objectively grade severity and disease progression of IC/PBS and other lower urinary track diseases.

### Example 3

### Chemokines, Cytokines, and Growth Factors are Elevated in Urine of Patients with Overactive Bladder (OAB)

Materials and Methods

Samples and Subjects

This study was carried out after the approval of the protocol and informed consent on the procedures by the University of Pittsburgh Institutional Review Board. A total of 25 midstream urine samples were collected after obtaining informed consent from eight asymptomatic healthy control subjects and 17 patients with idiopathic OAB. The diagnosis of OAB was based on a history of urgency, frequency with or without urgency/incontinence for more than 1 year. The control group included individuals with no active urologic disease or lower urinary tract symptoms at the time of urine collection. Patients with OAB were free of any neurologic disease and were on treatment prior to urine collection, including anti-muscarinic drugs, physical therapy, neuromodulation, and anti-depressants. Urinalysis was performed at the time of urine collection, and no pyuria or bacteria (suggestive of UTI) was identified. Freshly voided urine was immediately centrifuged at 2,400×g for 10 min, aliquoted, and frozen at −80° C. prior to cytokine analysis. In order to correct for hydration status, cytokine concentrations were normalized to, and expressed as, picograms per milligram of creatinine.

Luminex® Assays of Chemokines

Cytokines, chemokines, and growth factors were assayed using commercially available microspheres (Millipore, Billerica, Mass.) according to the manufacturer's instructions. Three groups of proteins were determined in the study: cytokines, including IL-5, IL-6, IL-10, IL-12p70/p40, IL-1 receptor antagonist (IL-1Ra), soluble IL-2 receptor a (sIL-136 2Ra), soluble fraction of the CD40 ligand (sCD40L); CC chemokines including monocyte chemotactic protein-1 (CCL2/CCL2), macrophage inflammatory proteins, MIP-1β/CCL4; and CXC chemokines including growth-related oncogene (GRO-α/CXCL1), CXCL10/IP-10, and growth factors such as epidermal growth factor (EGF). Microspheres of defined spectral properties conjugated to antibodies directed against urinary proteins were pipetted into a 96-well plate. The median fluorescence intensity of microspheres specific for each cytokine was recorded for each well and compared to the known standard values included in the manufacturer's kit. An automated immunoassay analyzer (Luminex® 100™ IS System, Luminex, Austin, Tex.) which is a continuous, random-access instrument that performs automated chemiluminescent immunoassays was used.

Statistical Analysis

Statistical differences in the median values of normalized protein levels between groups were determined by Mann-Whitney test using Graph-pad Prism version 4 (La Jolla, Calif.). The test assesses significant differences between groups without making assumption of normality, and differences were assessed at a two-tailed P10.05. The levels of each cytokine were normalized to, and expressed as, picograms per milligrams of creatinine (mean±SE) for both the control and OAB patients.

Results

The mean age of the subjects in the control group was 43.8±4.3 years (n=8) and 55.8±4.3 years in the OAB group (n=17). There were three males in the control and eight males in the OAB group, and the rest were females in both the groups. Analysis of urine from a single void of OAB patients revealed a significant elevation in a number of inflammation-associated proteins relative to asymptomatic controls (Table 1).

TABLE 1
Mean urine levels of protein (expressed as pg/mg) of
Creatinine) in asymptomatic controls and OAB patients

| Cytokine/Chemokine | Controls | OAB |
|---|---|---|
| sCD40L | 4.25 ± 2.99 | 43.5 ± 12.25* |
| IL-5 | 1.79 ± 1.22 | 13.05 ± 2.92* |
| GRO-α | 14.22 ± 8.92 | 106.32 ± 26.16* |
| MIP-1β | 4.74 ± 3.17 | 30.73 ± 8.87* |
| EGF | 2863 ± 376.3 | 17360.67 ± 3266* |
| IL-10 | 1.18 ± 0.71 | 7.33 ± 1.75* |
| IL-12p70/p40 | 2.04 ± 1.49 | 13.95 ± 4.15* |
| sIL-2Ra | 60.27 ± 34.02 | 222.2 ± 52.57* |
| CCL2 | 5.08 ± 4.12 | 144.33 ± 44.49* |
| IL-6 | 0.233 ± 0.06 | 7.51 ± 0.89 |
| IP-10 | 20.24 ± 9.43 | 13.48 ± 4.74 |
| IL-1Ra | 477.7 ± 180.1 | 673.2 ± 280.7 |

*p < 0.01

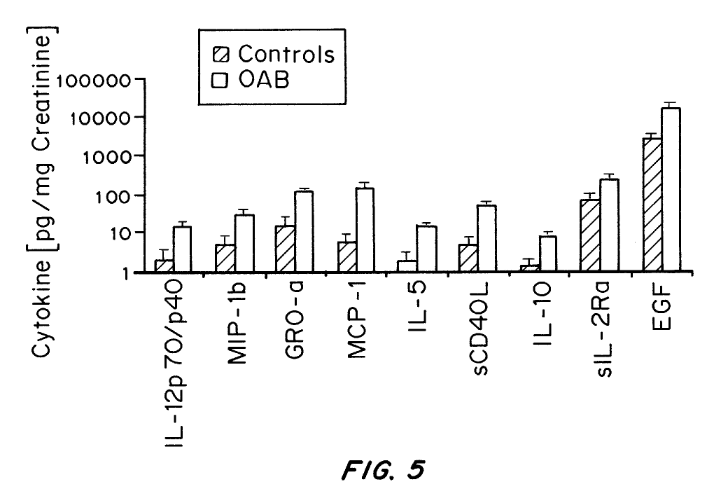

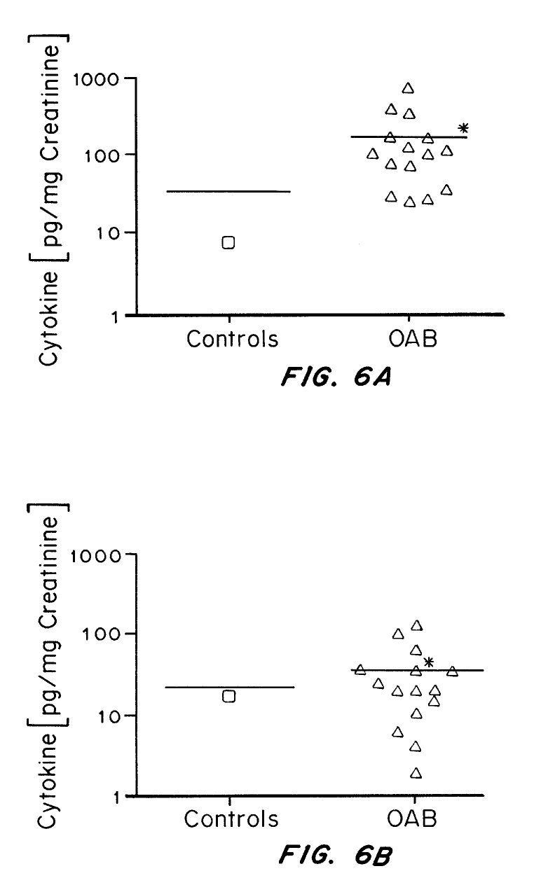

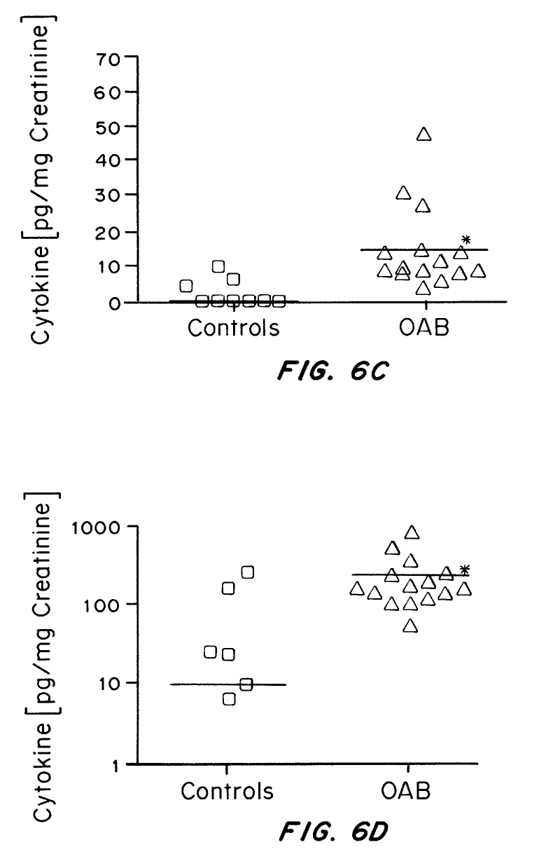

Analysis of urine through a 12-multiplex screen revealed significant elevation of seven key proteins in the urine of greater than 75% of the OAB patients relative to that in controls (*P\0.01; Table 1). Over tenfold elevation was noticed in the levels of CCL2, soluble fraction of the CD40 ligand (sCD40L) in urine obtained from OAB patients relative to controls (FIG. 5). At least fivefold elevations was detected in the OAB patients with regard to levels of MIP-1β, IL-12p70/p40, IL-5, EGF, and GRO-α compared to controls. Significant three fold elevations were also noticed in the urine levels of sIL-2Ra and IL-10 in the OAB group. Urine levels of IL-5 were above the detection limit of the assay in majority of control subjects, but MIP-1β and CCL2 were below the detection limit in the urine of subjects from control group (FIG. 6); notice the absence of log scale in panel for IL-5. The presence of similarly elevated levels of neutrophil-specific chemokines GRO-α and MIPI-10 suggests an infiltration of neutrophils in the bladder tissue of patients with OAB (Torrence, et al., Inflamm. Bowel Dis., 14(4):480-490 (2008)) EGF was the only biomarker present in the nanograms per milligrams of creatinine range in the urine of controls as well as in the diseased patients. The presence of EGF in urine is suggestive of tissue repair and fibrosis in OAB the patients, and may help in diagnosis and treatment of this disease.

Preclinical studies have shown that urine levels do not always reflect the high tissue concentrations for some of the pro-inflammatory cytokines (Smaldone, et al., Urology, 73(2): 421-26 (2009)). The urine chemokine profile of OAB patients indicates transcriptional regulation of their levels through nuclear factor kB (NF-kB) that is activated by pro-inflammatory cytokines (Bouchelouche, et al., Urology, 67:214-19 (2006)).

The results show a significant rise in the urine levels of the soluble form of CD40 (sCD40L) which is an important member of the TNF family produced by activated T cells (Ferroni, et al., Cardiovasc. Hematol. Disord Drug Targets, 8:194-202 (2008)). Increased serum levels of sCD40L have been associated with inflammatory states linked to hypertension. T_{H}2 infiltration may be indicated by the presence of IL-10 and IL-5. Given that elevated IL-10 is a hallmark of inflammation induced by sterile trauma, the results point to this mechanism, as well as a possible compensatory response to tissue inflammation (Bastian, et al., Eur. Surg. Res., 41:334-340 (2008), Bhangoo, et al., Mol. Pain, 3:38 (2007), Rossato, et al., Eur. J. Immunol., 37:3176-89 (2007)) in OAB. The finding of elevated IL-5, a T_{H}2 cytokine, further suggests a possible eosinophilic infiltration, which has been reported in the neurogenic bladder of spinal cord injury patients (Apostolidis, et al., Eur. Urol., 53:1245-53 (2008)).

Elevated levels of urine cytokines can be a manifestation of stressed lifestyle from OAB or sleep deprivation from nocturia and other troublesome symptoms of OAB. These cytokines and chemokines are known to affect the metabolism of neurotransmitters involved in the signaling of micturition reflex (White, et al., Curr. Opin., Anaesthesiol., 21:580-85 (2008), Kimball, et al., Neurogastroenerol. Motil., 19:390-400 (2007)). Therefore, it is possible that increased production of inflammatory cytokines may contribute to altered sensory processing in bladder. The up-regulation of inflammatory cytokines in OAB patients may occur as a result of an interaction of overactive parasympathetic (cholinergic) and peptidergic/sensory innervation of the bladder with local immune cells. Circulating hormones or locally released neurotransmitters and neuropeptides such as calcitonin gene-related peptide (CGRP) are known to affect proliferation and traffic of immune cells in the bladder, including the secretion of cytokines and the selection of both TH1 (IL-12p70/p40) and T_{H}2 cytokine (IL-5 and IL-10) responses (Bouchelouche, et al., J. Urol., 171:462-66 (2004)). In addition, CXCL1 chemokines have been associated with the release of CGRP and sympathetic remodeling (Qin, et al., J. Neurosci. Res. 82:51-62 (2005), Good, et al., J. Proteome Res., 6(12):4549-55 (2007)).

### Example 4

### OAB and IC/PBS can be Discriminated by Multivariate Data Modeling of Urinary Proteins

Materials and Methods

Mid stream urine samples were obtained from well-characterized 39 IC/PBS patients and 17 OAB patients after informed consent. The IC/PBS patients in the study cohort were predominantly females and their age was stochastically lower than that of OAB patients. Samples were analysed for 20 cytokines, chemokines and growth factors using Luminex® xMAP assay platform. The data on urinary proteins was analysed by univariate and multivariable analyses such as principal component analysis (PCA) to obtain distinct chemokine signatures of two diseases.

Results

The symptoms of overactive bladder (OAB) overlap with symptoms of Interstitial Cystitis/Painful Bladder Syndrome (IC/PBS) and there is an absence of diagnostic tools for objective classification. Out of the 20 inflammation associated proteins tested in urine only 15 were consistently detected categories. The PCA analysis identified principal drivers of variance and 90% of the variance in OAB group was explained by age, which is in agreement with higher prevalence of OAB in elderly population. No single protein among the 15 proteins consistently detected in urine could alone discriminate between the two disease groups. Using forward selection and Akaike information criterion for variable selection and model comparison by logistic regression, the addition of 3 inflammatory proteins, namely CCL2, IL-5 and eotaxin in the preferred model improved the prediction of IC/PBS over OAB. The model further predicts that rise in IL-5 and CCL2 increased the odds ratio for IC/PBS, wheras rise in eotaxin increased the odds ratio for OAB by 16% (p=0.04). Urine levels of IL-5, and IL-8 were stochastically higher in IC/PBS patients (p<0.0032) and levels of IP-10, IL-10 and eotaxin were higher in OAB patients (p<0.0447).

The differential in the urinary chemokine repertoire associated with OAB and IC/PBS indicate that mechanistically distinct inflammatory pathways underlie the two diseases. It is expected that analysis of urine in larger cohort of OAB and IC/PBS patients followed longitudinally will reduce the number of variables required in the predictive model and further optimize this model.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

We claim:

1. A method for diagnosing or assisting in the diagnosis of an urological disorder comprising
assaying an urine sample for concentrations of one or more biomarkers, wherein statistically different concentrations of the one or more of the biomarkers relative to a normal or asymptomatic control is indicative of an urological disorder.

2. The method of claim 1 for assessing the severity of an urological disorder comprising assaying an urine sample for concentrations of biomarkers correlated to the severity of an urological disorder.

3. The method of claim 1 for assessing the effectiveness of a treatment for an urological disorder comprising assaying an urine sample for biomarkers, wherein elevated concentrations of the biomarkers in the urine sample from the subject indicates the treatment for the urological disorder is ineffective or suboptimal.

4. The method of claim 1 further comprising correction of the samples for creatinine concentrations.

5. The method of claim 1 comprising performing statistical analysis of the assay results.

6. The method of claim 1 further comprising the step of recording or reporting a diagnosis of the urological disorder.

7. The method of claim 1 further comprising the step of generating a report.

8. The method of claim 1, wherein the urological disorder is interstitial cystitis/painful bladder syndrome and the biomarkers are chemokines relevant to chemotaxis of eosinophils, chemotaxis of monocytes, or activation of mast cells.

9. The method of claim 8 for facilitating the diagnosis of a subject for interstitial cystitis/painful bladder syndrome comprising measuring concentrations of at least one biomarker protein in an urine test sample, wherein the biomarker protein is selected form the group consisting of IL-8, CCL2, CCL4, and CCL11, wherein, a higher level of IL-8, CCL2, CCL4, and CCL11, in the urine test sample as compared to a reference level is indicative of interstitial cystitis/painful bladder syndrome.

10. The method of claim 8 wherein the chemokines are selected from the group consisting of CCL2, CCL4, and CCL11, and a level higher than normal is associated with urological disease or disorder.

11. The method of claim 1 wherein the urological disorder is overactive bladder syndrome and the biomarkers are selected from the group consisting of CCL2, CCL4 (MIP-1β), CCL11, CXCL1 (GRO-α), sCD40L, IL-12p70/p40, IL-5, sIL-2Rα, IL-6, IL-10, IL-8, EGF, and combinations thereof.

12. The method of claim 1 for facilitating the diagnosis of a subject for overactive bladder syndrome comprising measuring concentrations of at least one biomarker protein in an urine test sample, wherein the biomarker protein is selected form the group consisting of CCL2, CCL4 (MIP-1β), CXCL1 (GRO-α), sCD40L, IL-12p70/p40, IL-5, sIL-2Rα, IL-6, IL-10, and EGF, wherein, a higher level of CCL2, CCL4 (MIP-1β), CXCL1 (GRO-α), sCD40L, IL-12p70/p40, IL-5, IL-6, IL-10, and EGF, in the urine test sample as compared to a reference level is indicative of overactive bladder syndrome.

13. The method of claim 1 wherein at least two biomarkers are assayed.

14. The method of claim 1 wherein at least three biomarkers are assayed.

15. The method of claim 1 further comprising the step of recording or reporting the effectiveness of a treatment based on the concentrations of the biomarkers in the urine sample.

16. The method of claim 1, wherein the presence of the biomarker protein is detected using an antibody-based binding moiety which specifically binds to the biomarker protein.

17. The method of claim 1, wherein the concentration of the biomarker protein is measured by measuring the activity of the biomarker protein.

18. A kit designed for facilitating the diagnosis an urological disorder comprising
a means for detecting, in urine, one or more protein biomarkers;
a sample container for holding an urine sample;
reagents for measuring levels of the protein biomarker; and
instructions for use and reference values of the protein biomarker.

19. The kit of claim 18 wherein the urological disorder is interstitial cystitis/painful bladder syndrome and the biomarkers are selected from the group consisting of CCL2, CCL4, and CCL11.

20. The kit of claim 18 wherein the urological disorder is overactive bladder syndrome and the biomarkers are selected from the group consisting of CCL2, CCL4 (MIP-1β), CXCL1 (GRO-α), sCD40L, IL-12p70/p40, IL-5, sIL-2Rα, IL-6, IL-10, and EGF.

21. The kit of claim 18 comprising antibody based reagents.

22. A method for treating an urological disorder comprising administering an effective amount of an antagonist of a chemokine or cytokine relevant to an urological disorder to reduce inflammation in bladder relative to a control.

23. The method of claim 22 wherein the antagonist is selected from the group consisting of an antibody or antigen binding fragment thereof and inhibitory nucleic acids.

24. The method of claim 23 wherein the inhibitory nucleic acids are selected from the group consisting of siRNA, microRNA, antisense DNA, or a combination thereof specific for the chemokine or cytokine.
